(19) United States
(12) Patent Application Publication
(10) Pub. No.: US 2006/0282143 A1
(43) Pub. Date: Dec. 14, 2006

(54) Therapy pack holding device

(76) Inventor: Cindy D. Nunn, Fort Myers, FL (US)

Correspondence Address: CINDY D. NUNN, 15555 Caloosa Creek Circle, Ft Myers, FL, 33908, US

(21) Appl. No.: 11/150,954

(22) Filed: Jun. 13, 2005

(51) Int. Cl. A61F 7/00

(52) U.S. Cl. 607/114

(57) ABSTRACT

A therapy pack holding device (1) comprising a rear panel (5) and a front panel (7) secured together to form a pillowcase and a flap (8) having at least one pocket (2). The flap (8) is secured to the front panel (7) so as to permit a person to place his or her head in between the front panel (7) and the flap (8) so as to allow a therapy pack (4) to rest on a person's head. A second pocket (3) may be located proximate the bottom of the front panel (7) so as to permit a person to use a therapy pack (4) for the neck and/or shoulder area. The pockets (2) or (3) may be lined with a waterproof or water-absorbent material and the pockets (2) or (3) may have a fastening closure means (9) so as to maintain the therapy packs (4) within the pockets (2) or (3).

### BACKGROUND OF THE INVENTION

This invention relates to therapy pack holding devices, more particularly, a therapy pack holding device that allows a person to use one or more temperature therapy packs simultaneously on different areas of the head.

The use of temperature therapy packs for relief of aches and pains has long been in existence. Individuals use therapy packs for a variety of ailments, including headaches and shoulder/neck pain. The packs may be used when frozen or heated, depending upon the type of solution within the pack. Typically, however, ice packs are used as therapy packs as they are much more inexpensive than packs which may be heated. However, the use of frozen or cold packs has one severe disadvantage: condensation.

In order to avoid condensation from ruining couches, pillows or other fabrics, the cold or frozen pack may be placed on a towel or other piece of cloth to absorb the condensation. However, the use of a towel or cloth significantly reduces the amount of cold from the ice pack. In addition, positioning of the towel or cloth is oftentimes difficult as the towel or cloth may slide down, fold over or simply not stay in place.

Many individuals have found comfort and relief of aches and pains located on the back of the neck and/or upper shoulders by using pillows having a pocket for the insertion of a therapy pack. In addition, many individuals have found comfort and relief of headaches by positioning such therapy packs on the forehead. However, if the person moves his or her head slightly while the therapy pack is on the head, he or she could cause the pack to fall. Thus, a person is forced to lay or sit perfectly still while using the therapy pack on his or her head.

In addition, if a person suffers from a headache and pain in the neck or shoulders simultaneously, he or she must carefully position two therapy packs such that one is located on the head and one is located behind the neck and/or shoulders. Once again, the person must maintain a perfectly still position to keep the pack located on the head in place.

Thus, a need exists for a therapy pack holding device that permits a person to use a therapy pack on the head while permitting the person to move his or her head while simultaneously permitting a person to use a therapy pack on the neck and/or shoulders.

The relevant prior art includes the following references:

| Patent No. | | |
|---|---|---|
| (U.S. unless | | |
| stated otherwise) | Inventor | Issue/Publication Date |
| 4,887,326 | O′Brien et al. | Dec. 19, 1989 |
| 3,840,918 | Shave | Oct. 15, 1974 |
| 4,783,866 | Simmons, et al. | Nov. 15, 1988 |
| 6,668,400 | Nichols et al. | Dec. 30, 2003 |
| 5,948,013 | Swezey et al. | Sep. 07, 1999 |
| 5,916,088 | Gueli | Jun. 29, 1999 |
| 2004/0123392 | Stewart, III et al. | Jul. 01, 2004 |
| 6,804,845 | Stewart, III et al. | Oct. 19, 2004 |
| FR002656793 | Kruger et al. | Jul. 12, 1991 |

### SUMMARY OF THE INVENTION

The primary object of the present invention is to provide a therapy pack holding device that permits the use of therapy packs on the head and on the neck and/or shoulders simultaneously.

A further object of the present invention is to provide a therapy pack holding device that maintains a therapy pack on a person's head despite movement of the head.

An even further object of the present invention is to provide a therapy pack holding device that is easy to use.

Another object of the present invention is to provide a therapy pack holding device that absorbs condensation from frozen or cold therapy packs.

A further object of the present invention is to provide a therapy pack holding device that is washable.

An even further object of the present invention is to provide a therapy pack holding device that comfortably supports a person's head.

The present invention fulfills the above and other objects by providing a therapy pack holding device comprising a rear panel and a front panel secured together so as to form a pillowcase and a flap having at least one pocket. The flap is secured to the front panel so as to permit a person to place his or her head in between the front panel and the flap. Thus, a person may insert a therapy pack into the flap pocket so as to allow the therapy pack to rest on a person's head.

A second pocket may be located on the device such that when a therapy pack is inserted, the therapy pack is located behind a person's neck and/or shoulders. In the alternative, the therapy pack holding device may be in the form of a pillow wherein the pockets are attached directly to a pillow. The pockets may be lined with a waterproof or water-absorbent material and the pockets may have a fastening closing means so as to maintain the therapy packs within the pockets.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is a front plan view of the present invention in use;

FIG. 2 is a cross-sectional view along line 2-2 of a first embodiment of the present invention; and

FIG. 3 is a side view of a second embodiment of the present invention.

### DESCRIPTION OF THE PREFERRED EMBODIMENTS

For purposes of describing the preferred embodiment, the terminology used in reference to the numbered components in the drawings is as follows:

| | |
|---|---|
| 1. | therapy pack holding device, |
| | generally |
| 2. | first pocket |
| 3. | second pocket |
| 4. | therapy pack |
| 5. | rear panel |
| 6. | pillow |
| 7. | front panel |
| 8. | flap |
| 9. | first pocket fastening |
| | closure |
| | means |
| 10. | perimeter |
| 11. | opening |
| 12. | top edge |
| 13. | second pocket fastening |
| | closure means |
| 14. | bottom edge |

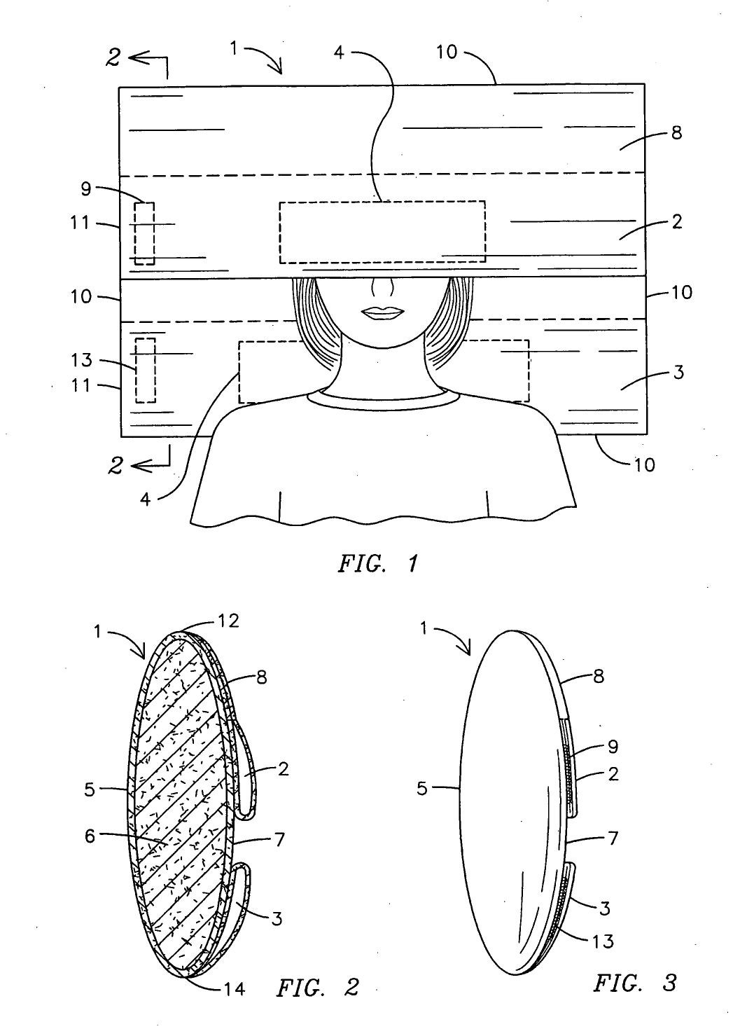

With reference to FIG. 1, a front plan view of the present invention in use is shown. The therapy holding device 1 having a perimeter 10, which is preferably rectangular in shape but may also be round or oval-like, comprises a first pocket 2 and a second pocket 3 for the accommodation of a plurality of therapy packs 4. To use the present invention, a person simply inserts a therapy pack 4 into the desired pocket 2 or 3 through an opening. Then, he or she may close the pocket 2 or 3 using a fastening closure means 9 or 13 located on an end of the pocketed therapy holding device 1. The fastening closure means may include, but are not limited to, hook and loop type material, buttons, snaps, zippers and the like.

Because the first pocket 2 is located on a flap 8, a person simply inserts his or her head behind the flap 8 such that the flap 8, and therapy pack 4 located in the first pocket 2 within the flap 8, are covering the desired portion of a person's face. If a person does not need to utilize the comfort of a therapy pack 4 on his or her face, then he or she may simply use the flap 8 to block light from shining into his or her eyes. The pockets 2 and 3 may be lined with a waterproof or water-absorbent material to resist or absorb condensation from frozen or cold therapy packs.

In FIG. 2, a cross-sectional view along line 2-2 of the present invention is shown. The therapy pack holding device 1 in the first embodiment is a pillowcase having a rear and front panel 5 and 7. A pillow 6 is inserted into the pillowcase so as to provide support for a user. The flap 8 portion is attached to the front panel 7 top edge 12 of the pillowcase. The second pocket 3 is also attached to the front panel 7, but along the bottom edge 14. The first pocket 2 is located within the flap 8.

Finally, FIG. 3 shows a side view of a second embodiment of the present invention. The second embodiment features a pillow having a rear and front panel 5 and 7. Similar to the first embodiment, the second embodiment of the present invention includes a first and second pocket 2 and 3 wherein the first pocket 2 is located within a flap 8. The pillow is filled with a predetermined material, preferably a soft, cushion-like material such as cotton, goose down feathers or the like.

The use of the present invention will allow a person to use a plurality of therapy packs 4 on different areas of the face and neck or shoulder area simultaneously.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not be considered limited to what is shown and described in the specification and drawings.

Having thus described my invention, I claim:

1. A therapy pack holding device comprising:
a rear panel having a predetermined perimeter;
a front panel having a same predetermined perimeter as said rear panel;
said front panel having a top edge and a bottom edge;
said rear panel perimeter being joined along a substantial portion of said front panel perimeter so as to form an opening for receiving a pillow;
an opening for receiving a pillow;
a flap secured to said front panel top edge;
at least one pocket in said flap; and
an opening in said at least one pocket for receiving a therapy pack.

2. The therapy pack holding device of claim 1 further comprising:
at least one pocket proximate the bottom edge of the front panel, said at least one pocket having an opening for receiving a therapy pack.

3. The therapy pack holding device of claim 1 wherein:
a fastening closure means is located proximate the opening of said at least one pocket.

4. The therapy pack holding device of claim 2 wherein:
a fastening closure means is located proximate the opening of said at least one pocket.

5. The therapy pack holding device of claim 1 wherein:
said at least one pocket is water-absorbent.

6. The therapy pack holding device of claim 2 wherein:
said at least one pocket is water-absorbent.

7. The therapy pack holding device of claim 1 wherein:
said at least one pocket is waterproof.

8. The therapy pack holding device of claim 2 wherein:
said at least one pocket is waterproof.

9. A therapy pack holding device comprising:
a rear panel having a predetermined perimeter;
a front panel having a same predetermined perimeter as said rear panel;
said front panel having a top edge and a bottom edge;
a predetermined material located between said rear and front panels;
said rear panel perimeter being joined entirely along said front panel perimeter so as to enclose said predetermined material between said rear panel and said front panel;
a flap secured to said front panel top edge;
at least one pocket in said flap; and
an opening in said at least one pocket for receiving a therapy pack.

10. The therapy pack holding device of claim 9 further comprising:
at least one pocket proximate the bottom edge of the front panel, said at least one pocket having an opening for receiving a therapy pack.

11. The therapy pack holding device of claim 9 wherein:
a fastening closure means is located proximate the opening of said at least one pocket.

12. The therapy pack holding device of claim 10 wherein:
a fastening means is located proximate the opening of said at least one pocket.

13. The therapy pack holding device of claim 9 wherein:
said at least one pocket is water-absorbent.

14. The therapy pack holding device of claim 10 wherein:
said at least one pocket is water-absorbent.

15. The therapy pack holding device of claim 9 wherein:
said at least one pocket is waterproof.

16. The therapy pack holding device of claim 10 wherein:
said at least one pocket is waterproof.

17. A therapy pack holding device comprising:
a rear panel having a predetermined perimeter;
a front panel having a same predetermined perimeter as said rear panel;
said front panel having a top edge and a bottom edge;
said rear panel perimeter being joined along a substantial portion of said front panel perimeter so as to form an opening for receiving a pillow;
an opening for receiving a pillow;
a flap secured to said front panel top edge;
at least one pocket on said flap;
an opening in said at least one pocket for receiving a therapy pack;
at least one pocket proximate the bottom edge of the front panel, said at least one pocket having an opening for receiving a therapy pack;
a fastening closure means located proximate the opening of said at least one pocket located on said flap; and
a fastening closure means is located proximate the opening of said at least one pocket located on said front panel.
